(19) United States
(12) Patent Application Publication
(10) Pub. No.: US 2025/0136921 A1
(43) Pub. Date: May 1, 2025

(54) FUNGAL BIOMASS, METHOD FOR THE PREPARATION AND USES THEREOF, AND EDIBLE COMPOSITIONS COMPRISING SAID FUNGAL BIOMASS

(71) Applicant: eniferBio Oy, Espoo (FI)

(72) Inventors: Simo Ellilä, Espoo (FI); Heikki Keskitalo, Oulu (FI); Anssi Rantasalo, Espoo (FI); Ville Pihlajaniemi, Helsinki (FI); Joosu Kuivanen, Tampere (FI)

(21) Appl. No.: 18/690,716

(22) PCT Filed: Sep. 9, 2022
(86) PCT No.: PCT/FI2022/050605

§ 371 Date: Mar. 8, 2024

(30) Foreign Application Priority Data
Sep. 10, 2021 (FI) 20215955

(51) Int. Cl. C12N 1/14; A23J 1/00; A23L 31/00

(52) U.S. Cl. CPC C12N 1/14; A23J 1/00; A23L 31/00

(57) ABSTRACT

A fungal biomass comprising one or more filamentous fungi each independently selected from the fungal family Trichocomaceae, wherein the fungal biomass has a crude protein content of at least 57% based on the total dry weight of the fungal biomass, and edible composition comprising the fungal biomass are disclosed. A method for the continuous preparation of a fungal biomass having a crude protein content of at least 57% based on the total dry weight of the fungal biomass is also disclosed, and further use of the fungal biomass and the edible composition as or in foodstuff is disclosed.

### CROSS-REFERENCE TO RELATED APPLICATIONS

This application is a national phase entry of and claims priority to International Patent Application No PCT/FI2022/050605 (filed 9 Sep. 2022), which claims priority to Finnish Patent Application No. 20215955 (filed 10 Sep. 2021), the entire disclosures of which are incorporated herein by reference.

### TECHNICAL FIELD

The present disclosure relates to fungal biomasses and methods for the preparation, edible compositions comprising said biomass, and uses thereof. More particularly, the present invention relates to a fungal biomass comprising filamentous fungi of the fungal family Trichocomaceae, wherein the fungal biomass has a high crude protein content.

### BACKGROUND

Biorefining becomes increasingly important with an increasing population and the need to combat climate change. The growing demand for food and sustainable food production are global challenges, and the lack of sustainably produced protein is a significant part of the problem. Production of protein source, which may be used as or in foodstuff, such as animal feed (fodder) and compound feed, has constantly increased in response to the rapidly increasing global demand. However, current production of many protein sources, such as meat, is not sustainable and/or ecological.

Fish feed containing fishmeal and fish oil as key components has widely been used since the 1970s. However, the production and comprehensive use of fishmeal are controversial since it may lead to environmental damage, depletion of ecosystems, and the collapse of local fisheries.

Soy protein concentrate (SPC) has commonly been used in modern fish feed in aquaculture. SPC is made from soybean and contains about 70% crude protein, which can be used as the protein source in fish feed. However, soy cultivation poses ecological challenges such as deforestation in South America and therefore, there is a demand for alternatives for SPC that are sustainable produced.

Proteinaceous substances suitable for use as fodder and foodstuffs have been manufactured by submerged aerobic cultures of species of mycelium-growing micro-organisms (patent FI44366B). Among processes disclosed in patent FI44366B are those wherein the micro-organism Paecilomyces varioti was cultivated in spruce calcium bisulphite spent liquor in a continuous cultivation process. A maximum protein content of 56.8% is disclosed when using Paecilomyces varioti.

In addition, agricultural waste has been used by the company BioTork as a substrate for algae and fungi to produce fish feed components.

Furthermore, biomass consisting of microalgae Nannochloropsis oculata and whole cells of DHA-rich Schizochytrium sp. for fish-free aquaculture feed has been disclosed (Sarker et al., Sci Rep. 2020; 10:19328).

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

One of the problems associated with known fungal biomasses useful as or in foodstuff is low crude protein content. It is therefore an object of the present invention to provide fungal biomasses having high protein content.

Other problem associated with known fungal biomasses useful as a protein source in foodstuff is higher production costs thereof compared to the production costs of plant proteins, especially soy protein, useful as protein sources in foodstuff. Therefore, a further object of the present invention is to provide methods for the preparation of fungal biomasses with lower production costs.

The invention is based on the realization that fungal biomasses comprising filamentous fungi of the fungal family Trichocomaceae may be prepared by methods of the invention, wherein these biomasses have a crude protein content of at least 57%.

The objects of the invention are achieved by fungal biomasses, and methods for the continuous preparation thereof, and edible compositions, and uses thereof that are characterized by what is stated in the independent claims. The preferred embodiments of the invention are disclosed in the dependent claims.

The present invention provides novel fungal biomasses comprising one or more filamentous fungi each independently selected from the fungal family Trichocomaceae, wherein the fungal biomass has a crude protein content of at least 57% based on the total dry weight of the fungal biomass.

The invention also relates to methods for the continuous preparation of a fungal biomass, comprising:
i) providing a feedstock comprising one or more dissolved carbon sources;
ii) optionally removing, at least partially, insoluble solids from the feedstock;
iii) optionally diluting or concentrating the feedstock;
iv) combining one or more filamentous fungi each independently selected from the fungal family Trichocomaceae with the feedstock;
v) cultivating the combined the one or more filamentous fungi and the feedstock under aerobic conditions to form the fungal biomass;
vi) collecting the formed fungal biomass from the feedstock when the formed fungal biomass has a crude protein content of at least 57%,
wherein the dilution rate is 0.3-0.5 h⁻¹.

The invention also provides edible compositions comprising fungal biomass as disclosed herein.

The invention also provides uses of a fungal biomass as disclosed herein or an edible composition as disclosed herein as or in foodstuff.

### DETAILED DESCRIPTION

“Optional” or “optionally” denotes that the subsequently described event or circumstance may but need not occur, and that the description includes instances where the event or circumstance occurs and instances in does which it not. “Comprises” or “comprising” denotes that the subsequently described feature(s) or act(s) may but need not include other feature(s) or act(s). It will further be understood that reference to ‘an’ item refers to one or more of those items.

The terms “fungal biomass” as used herein and hereafter refers to the mass of one or more fungal components, such as, but not limited to, proteins, amino acids, β-glucan, and chitin. It is to be understood that “fungal biomass” may be single-cell protein (SCP) or one or more fungal components, wherein at least one of the one or more fungal components is protein derived from the fungus/fungi. The terms “single-cell protein” as used herein and hereafter refers to edible unicellular fungus/fungi that comprise protein derived from said fungus/fungi. SCP may or may not be crude or refined unicellular fungus/fungi that may be dead or alive.

The terms “filamentous fungi” as used herein and hereafter refers to fungi that grow as tubular, elongated, and thread-like (filamentous) structures called hyphae.

The terms “fungal family Trichocomaceae” as used herein and hereafter refers to a family of fungi in the order Eurotiales. Examples of fungal genera of the fungal family Trichocomaceae include, but is not limited to, Paecilomyces, Gliocladium, Trichoderma, Byssochlamys, Spicaria, Aspergillus, Penicillium, Rasamsonia, Talaromyces, and Thermoascus. Examples of fungal species of the fungal family Trichocomaceae include, but is not limited to, Paecilomyces variotii, Paecilomyces puntonii, Gliocladium virens, Trichoderma viride, Byssochlamys nivea, Spicaria divaricata, Aspergillus niger, and Aspergillus oryzae.

The terms “crude protein content” as used herein and hereafter refers to the protein content of fungal biomass, composition, single-cell protein, or to which it may refer to and corresponds to the amount of nitrogen of said fungal biomass, composition, or single-cell protein multiplied by 6.25 (percentage of protein=6.25*N %) (dry weight basis of the fungal biomass, composition, or single-cell protein). Typically, crude protein content refers to the protein content of the fungal biomass based on the total dry weight of the fungal biomass. The crude protein content may be determined by methods known by the person skilled in the art, for example methods including, but not limited to, the Kjeldahl method and the Dumas method, preferably the Kjeldahl method. It is to be understood that the protein of the crude protein content of the fungal biomass refers to protein that originates from the one or more filamentous fungi. Furthermore, it is to be understood that an edible composition comprising fungal biomass as disclosed herein and hereafter may or may not contain further protein that may originate from other protein sources than the filamentous fungus as disclosed herein and hereafter.

The terms “dry weight” as used herein and hereafter refers to the mass of dried fungal biomass, composition, or single-cell protein, i.e. the mass of fungal biomass, composition, or single-cell protein excluding water.

The term “wt %” as used herein and hereafter refers to percentage by mass, i.e. the mass fraction (wᵢ) of the mass (mᵢ) to the total mass (mₜₒₜ) times a denominator of 100, i.e. the formula

wt %=wᵢ*100=(mᵢ/mₜₒₜ)*100,
wherein wᵢ=mass fraction, mᵢ=mass of substance, protein, or compound to which wt % refers to, and mₜₒₜ=the total mass of e.g. fungal biomass, feedstock, aqueous culture medium, or composition.

The Paecilomyces variotii strain KCL-24 is deposited with the recognised depositary institution VTT Culture Collection (VTTCC, Finland) with the accession number VTT D-211703.

The strain KCL-24 of fungus Paecilomyces variotii is deposited 27 Aug. 2021 under the Budapest Treaty by eniferBio Oy in the VTT Culture Collection (VTTCC, VTT Technical Research Centre of Finland Ltd, P.O. Box 1000, FI-02044 VTT, Finland) with an accession number VIT D-211703 (identification reference given by the depositor: KCL-24, PEKILO). The strain was received by the Depositary Authority on 27 Aug. 2021. The culture was confirmed viable on 30 Aug. 2021. The strain is characterized by the following morphological, culture and biochemical properties.

Morphological properties: a filamentous fungus, aerobic.

Culture properties: Forms white flat and powdery colonies on potato dextrose agar having from white to yellow color. Filamentous growth on submerged cultivation. Optimal cultivation temperature at 37° C.

Physiological properties: A filamentous fungus with a high specific growth rate (˜0.5 h⁻¹), ability to utilize many carbon sources, in particular, C5 sugars, acetate, formate and glycerol. High tolerance toward thermal biomass degradation products (furfural, formic acid, levulinic acid, 5-HMF and phenolics). Good filtration characteristics, high protein and beta-glucan content. It grows at a pH 3-7.5 at temperature 20-45° C. Uses ammonia, urea and amino acids as a sole nitrogen source.

Pathogenicity: the strain is not pathogenic. On the basis of the genome sequencing the strain is attributed to the genus Paecilomyces, variotii species. The method of cultivating the fungal strain Paecilomyces variotii KCL-24 may be carried out as described in the section EXAMPLES 1-6.

The term “fiber” as used herein and hereafter refers to the primary compounds of cell walls in e.g. fungi. Examples of fibers include, but are not limited to, chitin, and glucans.

The term “β-glucan” as used herein and hereafter refers to a group of β-D-glucose polysaccharides occurring in the cell walls of e.g. fungi. Typically, β-glucans form a linear backbone with 1-3 β-glycosidic bonds but vary with respect to molecular mass, solubility, viscosity, branching structure, and gelation properties. Common forms of β-glucans are those comprising D-glucose units with β-1,3 links. Fungal β-glucans may contain for example 1-6 side branches.

The term “ash” as used herein and hereafter refers to inorganics produced by combustion of biomass. Examples of inorganics comprised in biomass include, but are not limited to, calcium, iron, sodium, potassium, magnesium, phosphorous, and minerals thereof.

The term “fat” as used herein and hereafter refers to compounds that are typically lipophilic (i.e. non-water soluble) compounds. Fat may comprise one or more different lipophilic compound. Examples of compounds that fat may comprise include, but are not limited to, esters of fatty acids, fatty acids and salts thereof; mono-, di-, triglycerids, phospholipids, and/or cholesterol, or any combinations thereof.

The terms “water content” in combination with “wt %” as used herein and hereafter refers to water content percentage of the biomass or what it refers to, based on the total weight of the fungal biomass.

The term “feedstock” as used herein and hereafter refers to processed or unprocessed feedstock and fresh feedstock comprising one or more dissolved carbon sources and optionally one or more organic compounds and/or one or more inorganic compounds. It is to be understood that one or more filamentous fungi may be cultivated in the feedstock, i.e., the feedstock may be used as such as an aqueous culture medium for cultivating one or more filamentous fungi as disclosed herein and hereafter. The feedstock may be processed by removing, at least partially, insoluble solids from the feedstock, and/or diluting or concentrating the feedstock before, during, and/or after, or any combination thereof, cultivating the one or more filamentous fungi as disclosed herein and hereafter in the feedstock. It is to be understood that feedstock may also refer to used feedstock (e.g. used aqueous culture medium), wherein one or more filamentous fungi as disclosed herein and hereafter has been cultivated and therefore, the amount of one or more dissolved carbon sources of the used feedstock may be lower than in feedstock, wherein one or more filamentous fungi as disclosed herein and hereafter has not been cultivated (i.e. fresh feedstock), i.e. the composition of the feedstock may vary during a method as disclosed herein and hereafter. For example, before the one or more filamentous fungi is cultivated in the feedstock the feedstock may comprise a higher content of the one or more dissolved carbon sources than during and/or after said cultivation of the one or more filamentous fungi. It is to be understood that the feedstock may be an aqueous culture medium. “Fresh feedstock” refers to feedstock comprising one or more carbon sources, wherein one or more filamentous fungi has not been cultivated in, or to feedstock comprising higher content of one or more carbon sources than feedstock comprising one or more carbon sources, wherein one or more filamentous fungi has been cultivated in.

The terms “aqueous culture medium” as used herein and hereafter refers to growth medium comprising water and one or more carbon sources and optionally one or more organic compounds and/or one or more inorganic compounds, wherein the aqueous culture medium allows and/or promotes growth and/or cell proliferation of microorganisms, such as fungus as disclosed herein and hereafter. It is to be understood that the aqueous culture medium may be a feedstock as disclosed herein and hereafter.

Examples of organic compounds include, but are not limited to, carbohydrates, carbohydrate derivatives, sugars, polyols, carboxylic acids, amino acids, alcohols, esters of carboxylic acids, antifoaming agents such as Struktol J673A (alkoxylated fatty acid esters on vegetable base from Schill+Seilacher GmbH), and any combinations thereof.

Examples of inorganic compounds include, but are not limited to, nitrogen containing compounds such as NH₄OH, (NH₄)₂SO₄, CH₄N₂O, (NH₄)₂HPO₄; and phosphorous containing compounds such as H₃PO₄, and phosphates; KCl, MgSO₄, Fe₂(SO₄)₃, ZnSO₄, CuSO₄, MnSO₄, HCl, Vogel's trace elements, and any hydrates and combinations thereof. Vogels's trace elements comprises citric acid (e.g. 5 wt % in H₂O), ZnSO₄ (e.g. 5 wt % in H₂O), Fe(NH₄)₂(SO₄)₂ (e.g. 1 wt % in H₂O), CuSO₄ (e.g. 0.25 wt % in H₂O), MnSO₄ (e.g. 0.05 wt % in H₂O), H₃BO₃ (e.g. 0.05 wt % in H₂O), and Na₂MoO₄ (e.g. 0.05 wt % in H₂O). A person skilled in the art is aware that the aqueous culture medium (and the feedstock) may further trace comprise elements (inorganic trace substances) that may be beneficial for the growth and/or cell proliferation of the one or more filamentous fungi. It is to be understood that the aqueous culture medium may be the feedstock as defined herein and hereafter.

Examples of feedstocks and aqueous culture media include, but are not limited to, stillage, thin stillage, vinasse, molasses, spent sulphite liquor, prehydrolysis liquor, food industry processing waste, and other biorefinery by-products, or any mixture or combination thereof. Said feedstock may be or may not be a clarified feedstock, i.e. a feedstock, which has been treated in order to remove, at least partially, insoluble or suspended solids thereof.

“Thin stillage” as used herein and hereafter refers to stillage (from ethanol production using e.g. corn or wheat), which solids have been partially removed by e.g. centrifugation and therefore, it is to be understood that thin stillage comprises insoluble solids.

“Vinasse” as used herein and hereafter refers to a by-product of the sugar and ethanol industry. Vinasse is obtained as a by-product of the distillation step subsequent to fermentation of carbohydrates obtained from different sources of saccharides materials (e.g. sugarcane and beet), starchy materials (e.g. maize, wheat, rice, cassava, and oat), and lignocellulosic materials (e.g. sugarcane bagasse, straw, and wood, among others).

“Molasses” as used herein and hereafter refers to a product resulting from refining sugarcane or sugar beets into sugar.

“Spent sulphite liquor” as used herein and hereafter refers to spent cooking liquor from sulfite pulping and is also called brown liquor, red liquor, thick liquor and sulfite liquor.

“Prehydrolysis liquor” as used herein and hereafter refers to a liquor from the pre-hydrolysis stage in the dissolving pulp production process, that is rich in one or more dissolved carbon sources derived from hemicellulose, such as sugars and carboxylic acids.

Examples of food industry processing waste include, but are not limited to, brewery wastewater from beer brewing, pot ale and spent lees from the manufacture of whisky, potato processing waste, dairy subproducts such as whey permeate and delactosed permeate. Examples of other biorefinery by-products include, but are not limited to, corn steep liquor, soy molasses, and palm mill oil effluent (POME).

The terms “solids that are insoluble” and “insoluble solids” as used herein and hereafter refers to any nondissolved organic- and inorganic compounds, carbon sources, and chemical elements of feedstocks and/or aqueous culture media, i.e. organic- and inorganic compounds, carbon sources, and chemical elements that are not dissolved. Examples of solids that are insoluble in a feedstock include, but are not limited to, insoluble solids of biorefinery by-products, such as, but not limited to, cellulose; insoluble biomaterial, insoluble solids and suspended solids originating from fermentation process, thin stillage, vinasse, spent sulphite liquor, prehydrolysis liquor, and food industry processing waste, such as, but not limited to, CaSO₄. Solids that are insoluble in a feedstock may also refer to solids, such as carbon sources, that are soluble in a feedstock to a certain concentration, but the feedstock is saturated by said solid and therefore, at least a part of the solid is in the form of a precipitate or suspension. In addition, it is to be understood that “solids that are insoluble” and “insoluble solids” exclude fungal biomass as disclosed herein and hereafter.

The terms “fungal biomass is essentially free from solids that are insoluble” as used herein and hereafter refers to biomasses that contains no, very low, or low amounts of solids originating from solids that are insoluble in a feedstock and/or an aqueous culture medium used to cultivate the filamentous fungi as disclosed herein and hereafter to form the fungal biomass, wherein the solids may be any organic- and/or inorganic compounds and/or chemical elements that are insoluble in the feedstock. In this context it is to be understood that the fungal biomasses may comprise solids that are insoluble in a feedstock, however, these insoluble solids formed during may have the proliferation of the filamentous fungus. “Fungal biomass is essentially free from solids that are insoluble in a feedstock” may be fungal biomass containing <1.0 wt %, preferably <0.5 wt %, more preferably 0 wt %, of solids that are insoluble in a feedstock comprising one or more dissolved carbon sources, based on the total weight of the feedstock, and said solids that are insoluble may have a size of >1.0 μm, preferably >0.5 μm, more preferably >0.2 μm. It is to be understood that a fungal biomass having both a smaller content and a smaller size of solids that are insoluble in a feedstock is desirable. “Essentially free” in this context may also mean that solids that may be insoluble in a feedstock may have been removed, at least partially, from the feedstock, which may be used in a method for the continuous preparation of a fungal biomass as disclosed herein and hereafter to form the fungal biomass, and therefore, the fungal biomass (formed by cultivating the combined one or more filamentous fungi and the feedstock) may not contain the removed solids. Preferably, solids that are insoluble in a feedstock and with a size of at least 1.0 μm, preferably at least 0.5 μm, more preferably at least 0.2 μm, have been removed, at least partially, from the feedstock, preferably >50%, >70%, >80%, >90%, >95%, >97%, or >99%, of the solids that are insoluble in a feedstock have been removed from the feedstock. More preferably, “fungal biomass is essentially free from solids that are insoluble in a feedstock” refers to biomass that contains no solids that are insoluble in a feedstock comprising one or more dissolved carbon sources.

The term “carbon sources” as used herein and hereafter refers to molecules used by an organism as the source of carbon for building its biomass. Examples of carbon sources include, but are not limited to, carbohydrates, carbohydrate derivatives, polyols, carboxylic acids, esters of carboxylic acids, nucleotides, and alcohols.

The term “carbohydrates” as used herein and hereafter refers to compounds comprising oxygen, hydrogen, and at least one carbon. Examples of carbohydrates include, but are not limited to, sugars, oligosaccharides, and polysaccharides such as glucose, mannose, xylose, arabinose, galactose, fructose, sucrose, maltose, isomaltulose, trehalose, lactose, maltotriose, maltodextrins, xylooligosaccharides (XOS), raffinose, stachyose, and fructo-oligosaccharides.

The term “polyols” as used herein and hereafter refers to compounds comprising at least two hydroxyl groups. Examples of polyols include, but are not limited to, glycerol, mannitol, and sorbitol.

The terms “carboxylic acids” as used herein and hereafter refers to compounds comprising at least one a carboxyl group. Examples of carboxylic acids include, but are not limited to, formic acid, acetic acid, lactic acid, propionic acid, sugar acids, and amino acids.

The term “esters of carboxylic acids” as used herein and hereafter refers to compounds derived from carboxylic acids, wherein at least one OH-group has been replaced by an alkoxy group. Examples of alkoxy groups include, but are not limited to, methoxy, ethoxy, and alkoxy groups derived from carbohydrates and carbohydrate derivatives.

The term “alcohols” as used herein and hereafter refers to compounds comprising a hydroxyl group. Examples of alcohols include, but are not limited to, methanol, ethanol, and ethylene glycol.

The terms “carbohydrate derivatives” as used herein and hereafter refers to carbohydrates that have been modified with one or more substituents. Carbohydrate derivatives may also be carbohydrates or carbohydrate analogues that further comprise one or more heteroatom each independently selected from the group consisting of N, S, and Se. Examples of carbohydrate derivatives include, but are not limited to, glycosides, glycosylamines, N-acetylglucosamine, sugar phosphates, and esters of carbohydrates such as carbohydrate acetates. It is to be understood that the carbohydrate derivative may or may not function as a precursor material or an intermediate in the biosynthesis of, or conversion to, a carbohydrate.

The term “edible” as used herein and hereafter refers to any substance, fungal biomass, and composition that is considered safe for animals and/or humans to eat such as, but not limited to, foodstuff.

The term “foodstuff” as used herein and hereafter refers to food for consumption of humans and/or animals such as, but not limited to, fish, shrimps, crayfish, prawns, and domestic animals such as pig, sheep, cattle, and chicken. It is to be understood that foodstuff includes food for consumption of humans, compound feed, fodder, and animal feed, such as, but not limited to, fish feed, and aquafeed.

The term “continuous preparation” as used herein and hereafter refers to continuous cultivation of one or more filamentous fungi to form fungal biomass. Typically, during continuous preparation fresh feedstock is continuously added to a bioreactor containing one or more filamentous fungi and feedstock, while fungal biomass and often also culture liquid comprising left over nutrients (e.g. one or more dissolved carbon sources) and/or metabolic end products are continuously removed at the same rate as the addition of the fresh feedstock to keep the culture volume constant. By changing the rate with which fresh feedstock is added to the bioreactor (i.e. the dilution rate) the specific growth rate of the fungus/fungi may be controlled. Examples of bioreactors include, but are not limited to, stirred tank, airlift, and any system suitable for cultivation of one or more filamentous fungi to form fungal biomass. The term “removing, at least partially, insoluble solids from the feedstock” and “removed” in the same context as used herein and hereafter refers to any method of removing insoluble solids, wherein the amount of insoluble solids decreases. Examples of removing include, but are not limited to, filtering, clarification, decantating, settling, centrifugating, and screening.

The term “diluting” as used herein and hereafter refers to any method of decreasing the concentration of one or more dissolved carbon sources of the feedstock. Examples of diluting include, but are not limited to, adding water, solvent, one or more organic compounds, provided that the one or more organic compounds are selected from other organic compounds than said one or more dissolved carbon sources; and/or inorganic or combinations thereof, and another feedstock to the feedstock.

The term “concentrating” as used herein and hereafter refers to any method of increasing the concentration of the one or more dissolved carbon sources of the feedstock and/or aqueous culture medium. Examples of concentrating include, but are not limited to, addition of carbon sources to the feedstock and/or aqueous culture medium, removing such as evaporating water and/or solvent from the feedstock, and dialysis of the feedstock and/or aqueous culture medium.

The term “cultivating” as used herein and hereafter refers to the process of growth and/or cell proliferation of microorganisms, such as fungus.

The terms “aerobic conditions” as used herein and hereafter refers to conditions that include oxygen.

The terms “collecting the formed fungal biomass from the feedstock” as used herein and hereafter refers to any method by which formed fungal biomass may be collected from feedstock. Examples of collecting the formed fungal biomass from feedstock include, but are not limited to, filtering, decantating, settling, centrifugating, and screening.

The terms “dilution rate” used herein and hereafter refers to the rate of feedstock exchange in methods of continuous preparation of fungal biomass as disclosed herein and hereafter. During continuous preparation, when formed fungal biomass is continuously collected from the feedstock, fresh feedstock is continuously added at the same rate as collecting the formed fungal biomass to keep the culture volume constant. By changing the rate fresh feedstock is added to the bioreactor (i.e. the dilution rate (D)), the specific growth rate of the fungus/fungi may be controlled. The dilution rate (D) may be at steady state equal to the specific growth rate (u) of the one or more filamentous fungi. Steady state refers to the situation, wherein growth occurs at a constant specific growth rate and all culture parameters remain constant (culture volume, dissolved oxygen concentration, nutrient and product concentrations, pH, cell density, etc.). The dilution rate (D) is defined as flow of feedstock per unit of time (F) over volume (V) of filamentous fungi culture (the volume (V) of filamentous fungi culture refers to the volume of feedstock and one or more filamentous fungi in e.g. the bioreactor):

$$D = F / V .$$

The term “drying” as used herein and hereafter refers to any method of removing water (dewatering) or another solvent. Examples of drying include, but are not limited to, drying using hot air (with a temperature of, for example, 40-80° C., 50-70° C., or 50° C.), filtering, freeze drying, indirect or contact drying (such as heating through a hot wall) such as drum drying and vacuum drying; natural air drying. Examples of drying using hot air include, but is not limited to, the use of conveyor dryer, fluid bed dryer, flash dryer, and ring dryer.

The terms “cell density” as used herein and hereafter refers to the number or mass of cells per unit volume. Cell density may be denoted as viable cell density which is the number or mass of living cells per unit volume.

The terms “food ingredient” as used herein and hereafter refers to any substance and composition that is considered safe for animals and/or humans to eat. Food ingredients may be added to e.g. foodstuff (food for consumption of humans, animal feed, aquafeed, fodder, compound feed), and fungal biomasses and edible compositions as disclosed herein and hereafter to achieve a desired effect. Examples of food ingredients include, but are not limited to, fishmeal, fish oil, fish feed, soy protein concentrate, soy, soybeans, wheat protein, pea protein, soy protein isolate, wheat protein isolate, pea protein isolate, protein sources, food additives such as acidulants, acidity regulators, anticaking agents, antifoaming and foaming agents, antioxidants, bulking agents, food coloring, fortifying agents, color retention agents, emulsifiers, flavors, flavor enhancers, flour treatment agents, glazing agents, humectants, tracer gas, preservatives, stabilizers, sweeteners, and thickeners; corn, grain sorghum, oats, rye, barley; flours such as wheat, rye, farina, and meal flours; dairy products such as milk, yoghurt, curdled milk (soured milk), and cheeses such as cottage cheese. It is to be understood that a fungal biomass as disclosed herein and hereafter may be a food ingredient in foodstuff.

The term “fish feed” as used herein and hereafter refers to foodstuff for fish. Conventional fish feed may contain fishmeal, fish oil, and/or SPC.

The term “fishmeal” as used herein and hereafter refers to a food ingredient that fish feed may comprise, wherein fishmeal is mostly made from fish or parts thereof. Typically, fishmeal is food used to feed fish. Fishmeal may be made by cooking, pressing, drying, and/or grinding of fish or fish waste into a solid, wherein at least partially the water and some or all of the oil is removed.

The term “aquafeed” as used herein and hereafter refers to foodstuff for aquatic organisms such as, but not limited to, fish, shrimps, crayfish, and prawns.

In one aspect is disclosed a novel fungal biomass comprising one or more filamentous fungi each independently selected from the fungal family Trichocomaceae, wherein the fungal biomass has a crude protein content of at least 57% based on the total dry weight of the fungal biomass. A crude protein content of at least 57% is considered as a high protein content. A fungal biomass having a crude protein content of at least 57% is beneficial, since the fungal biomass may be used as an alternative to soy protein or SPC of conventional fish feed or aquafeed. Alternatively, the fungal biomass as disclosed herein and hereafter may be used to, at least partially, replace soy protein or SPC of conventional fish feed and aquafeed, and therefore, the fungal biomass is an ecological alternative to soy protein and SPC. Due to high crude protein content of the fungal biomass as disclosed herein and hereafter, foodstuff, preferably aquafeed or fish feed, comprising fungal biomass as disclosed herein and hereafter has lower production cost than foodstuff comprising conventional biomasses.

Alternatively, the fungal biomass has a crude protein content of 57-99% based on the total dry weight of the fungal biomass. Alternatively, the fungal biomass has a crude protein content of 57-94%, 57-91%, 57-87%, 57-83%, 57-79%, 57-75%, 57-73%, 60-70%, 60-73%, 60-77%, 60-81%, 60-85%, 60-89%, 60-93%, 60-95%, or 60-99%. Alternatively, the fungal biomass has a crude protein content of 63-99%, 63-95%, 63-91%, 63-87%, 63-83%, 63-79%, 63-75%, 63-73%, 63-70%, 66-99%, 66-95%, 66-91%, 66-87%, 66-83%, 66-79%, 66-75%, 66-73%, or 66-70%. Additionally, or alternatively, the crude protein content of the fungal biomass is at least 61%, or 61-70%, 61-73%, 61-77%, 61-81%, 61-85%, 60-89%, 60-93%, 60-95%, or 60-99%, based on the total dry weight of the fungal biomass. These fungal biomasses are advantageous since they may be used as alternatives to soy protein or SPC of conventional fish feed or aquafeed.

Alternatively, the fungal biomass has a crude protein content of 60-73% based on the total dry weight of the fungal biomass.

Alternatively, the one or more filamentous fungi is each independently selected from the fungal genera Paecilomyces, Aspergillus, Penicillium, Rasamsonia, Talaromyces, and Thermoascus. Fungal biomasses of these fungal genera are suitable as or in foodstuff.

Alternatively, the one or more filamentous fungi is each independently selected from the fungal species Paecilomyces variotii, Paecilomyces puntonii, Gliocladium virens, Trichoderma viride, Byssochlamys nivea, Spicaria divaricata, Aspergillus niger, and Aspergillus oryzae. Fungal biomasses comprising fungal species Paecilomyces variotii, Paecilomyces puntonii, Aspergillus niger, and/or Aspergillus oryzae, or any combination thereof, are especially suitable as or in foodstuff.

Alternatively, the one or more filamentous fungi comprises or is Paecilomyces variotii strain KCL-24, preferably is Paecilomyces variotii strain KCL-24. It has surprisingly been found that fungal biomass comprising or consisting of Paecilomyces variotii strain KCL-24 has a high crude protein content, i.e. a crude protein content of at least 57% based on the total dry weight of the fungal biomass. In addition, Paecilomyces variotii KCL-24 has a suitable amino acid composition for foodstuff, a good protein digestibility in animals, and lack mycotoxins. Alternatively, or additionally, fungal biomasses comprising Paecilomyces variotii strain KCL-24 as disclosed herein and hereafter have a crude protein content of at least 60%, 60-70%, 60-73%, 60-77%, 60-81%, 60-85%, 60-89%, 60-93%, 60-95%, or 60-99% based on the total dry weight of the fungal biomass. Alternatively, the fungal biomass comprising Paecilomyces variotii strain KCL-24 has a crude protein content of 63-99%, 63-95%, 63-91%, 63-87%, 63-83%, 63-79%, 63-75%, 63-73%, 63-70%, 66-99%, 66-95%, 66-91%, 66-87%, 66-83%, 66-79%, 66-75%, 66-73%, or 66-70%.

Alternatively, the fungal biomass comprises Paecilomyces variotii strain KCL-24, wherein the fungal biomass has a crude protein content of 60-73% based on the total dry weight of the fungal biomass.

Alternatively, the fungal biomass consists Paecilomyces variotii strain KCL-24, wherein the fungal biomass has a crude protein content of 60-73% based on the total dry weight of the fungal biomass.

Additionally, or alternatively, the total amino acid content of the fungal biomass is at least 48 g/100 g fungal biomass, preferably at least 52 g/100 g fungal biomass.

Additionally, or alternatively, the fungal biomass comprises or consists of Aspergillus oryzae, in particular strain number D-88355T, wherein the crude protein content of the fungal biomass is at least 61%, or 61-70%, 61-73%, 61-77%, 61-81%, 61-85%, 60-89%, 60-93%, 60-95%, or 60-99%, based on the total dry weight of the fungal biomass. These fungal biomasses are advantageous since they may be used as alternatives to soy protein or SPC of conventional fish feed or aquafeed.

Alternatively, or additionally, the fungal biomass comprises 0.5-10 wt % water, a total fiber content of 10-35 wt %, a β-glucan content of 10-25 wt %, 1-10 wt % ash, and 1-10 wt % fat based on the total weight of the fungal biomass. These fungal biomasses have beneficial fish immunostimulant properties and are beneficial for fish health.

In embodiments, the fungal biomasses consist of Paecilomyces variotii strain KCL-24, wherein the crude protein content is selected from 60-73%, 63-73%, 65-73%, 63-70%, and 65-70%, based on the total dry weight of the fungal biomasses.

In embodiments, the fungal biomasses comprise 0.5-10 wt % water and 90-99.5 wt % Paecilomyces variotii strain KCL-24 based on the total weight of the fungal biomass, wherein the crude protein content of the fungal biomasses is selected from 60-73%, 63-73%, 65-73%, 63-70%, and 65-70%, based on the total dry weight of the fungal biomasses.

Alternatively, or additionally, the fungal biomass has a water content of 3-8 wt %, preferably 4-7 wt %, based on the total weight of the fungal biomass. The fungal biomasses have a preferred protein digestibility. In addition, the fungal biomasses have a preferred shelf-life and enable cost-effective transportation due to the low content of water. Furthermore, the water content of the fungal biomasses enables effective use in apparatus for preparation of foodstuff, preferably animal feed and aquafeed.

Alternatively, or additionally, the fungal biomass is essentially free from solids that are insoluble in a feedstock comprising one or more dissolved carbon sources. Alternatively, or additionally, the fungal biomass contains <1.0 wt %, preferably <0.5 wt %, more preferably 0 wt %, of solids that are insoluble in a feedstock, based on the total weight of the feedstock comprising one or more dissolved carbon sources, preferably said solids that are insoluble may have a size of >1.0 μm, preferably >0.5 μm, more preferably >0.2 μm. These fungal biomasses may be less toxic to aquatic organisms and humans.

Alternatively, or additionally, the fungal biomass is edible. Therefore, these fungal biomasses are safe for animals and/or humans to eat and may be used as or in foodstuff such as, but not limited to, food for consumption of humans, animal feed, fodder, and/or compound feed, preferably as or in fish feed and/or aquafeed. Preferably, the foodstuff is fish feed or aquafeed.

Alternatively, or additionally, the fungal biomass is foodstuff. Alternatively, the fungal biomass is animal feed. Preferably, the animal feed is fish feed or aquafeed.

Alternatively, a fungal biomass comprising one or more filamentous fungi each independently selected from the fungal family Trichocomaceae, wherein the fungal biomass has a crude protein content of at least 57% based on the total dry weight of the fungal biomass, is obtainable by a method for the continuous preparation of the fungal biomass, comprising:
i) providing a feedstock comprising one or more dissolved carbon sources;
ii) optionally removing, at least partially, insoluble solids from the feedstock;
iii) optionally diluting or concentrating the feedstock;
iv) combining one or more filamentous fungi each independently selected from the fungal family Trichocomaceae with the feedstock;
v) cultivating the combined the one or more filamentous fungi and the feedstock under aerobic conditions to form the fungal biomass;
vi) collecting the formed fungal biomass from the feedstock when the formed fungal biomass has a crude protein content of at least 57% based on the total dry weight of the fungal biomass,
wherein the dilution rate is 0.3-0.5 h⁻¹.

Preferably, the feedstock comprising one or more dissolved carbon sources is essentially free from insoluble solids. Alternatively, or additionally, the the feedstock contains <1.0 wt %, preferably <0.5 wt %, more preferably 0 wt %, of solids that are insoluble in the feedstock, based on the total weight of the feedstock comprising one or more dissolved carbon sources, preferably said solids that are insoluble may have a size of >1.0 μm, preferably >0.5 μm, more preferably >0.2 μm. Even more preferably, the feedstock is free from insoluble solids.

Additionally, or alternatively, the one or more filamentous fungi is Paecilomyces variotii strain KCL-24. Additionally, or alternatively, the formed fungal biomass has a crude protein content that is selected from 60-73%, 63-73%, 65-73%, 63-70%, and 65-70%, based on the total dry weight of the fungal biomasses.

In one aspect is disclosed a method for the continuous preparation of a fungal biomass, comprising:
i) providing a feedstock comprising one or more dissolved carbon sources;
ii) optionally removing, at least partially, insoluble solids from the feedstock;
iii) optionally diluting or concentrating the feedstock;
iv) combining one or more filamentous fungi each independently selected from the fungal family Trichocomaceae with the feedstock;
v) cultivating the combined the one or more filamentous fungi and the feedstock under aerobic conditions to form the fungal biomass;
vi) collecting the formed fungal biomass from the feedstock when the formed fungal biomass has a crude protein content of at least 57% based on the total dry weight of the fungal biomass,
wherein the dilution rate is 0.3-0.5 h⁻¹.

Methods for the continuous preparation of a fungal biomass as disclosed herein and hereafter enables the preparation of fungal biomasses with high cude protein content. A crude protein content of at least 57% based on the total dry weight of the fungal biomass is considered a high protein content. Additionally, the methods enable an efficient preparation of fungal biomasses from various feedstocks comprising one or more dissolved carbon sources. A person skilled in the art understands that dilution rate includes fresh feedstock is continuously added at the same rate as continuously collecting the formed fungal biomass to keep the volume of the combined one or more filamentous fungi and the feedstock in iv), i.e., the culture volume, constant. It has surprisingly been found that dilution rates of 0.3-0.5 h⁻¹ in the continuous preparation of a fungal biomass according to methods disclosed herein and hereafter form fungal biomasses that have a crude protein content of at least 57% based on the total dry weight of the fungal biomass. Due to high crude protein content formed by the continuous preparation method, the production cost of protein of fungal biomass formed by the method is lower compared to conventional methods for preparing protein of fungal biomasses that result in lower crude protein contents of the biomasses. In addition, the continuous preparation combined with the dilution rate (h⁻¹) according to a method as disclosed herein and hereafter provides both an increased productivity and crude protein content of the formed biomass. Methods for continuous preparation of fungal biomasses as disclosed herein and hereafter enables continuous collecting, e.g. by filtrating, of the formed fungal biomass and, therefore, compared to e.g. batch process of fungal biomass, provides an increased productivity since interruptions in the preparation of fungal biomass may be avoided. Additionally, methods as disclosed herein and hereafter comprising the use of filamentous fungus, in particular Paecilomyces variotii strain KCL-24, may enable easier and/or cheaper collecting of the formed fungal biomass, since compared to conventional methods for preparation of fungal biomass utilizing centrifugation to collect formed biomass, the collecting the formed fungal biomass in the methods as disclosed herein and hereafter may be performed by simply filtering the formed fungal biomass from the feedstock. This may have a favorable impact on productivity.

Additionally, or alternatively, the v) cultivating the combined one or more filamentous fungi and the feedstock under aerobic conditions to form the fungal biomass is performed in a bioreactor. In embodiments, the bioreactor is selected from a stirred tank, airlift, and any system suitable for cultivation of one or more filamentous fungi to form the fungal biomass.

Additionally, or alternatively, the feedstock comprises water and one or more dissolved carbon sources.

Additionally, or alternatively, the feedstock is essentially free from insoluble solids that have a size of at least 1.0 μm, preferably at least 0.5 μm, more preferably at least 0.2 μm. Alternatively, or additionally, the feedstock contains <1.0 wt %, preferably <0.5 wt %, more preferably 0 wt %, of solids that are insoluble in the feedstock, based on the total weight of the feedstock comprising one or more dissolved carbon sources, preferably said solids that are insoluble may have a size of >1.0 μm, preferably >0.5 μm, more preferably >0.2 μm. It has surprisingly been found that when the feedstock is essentially free from insoluble solids high crude protein content of fungal biomass is formed in the methods disclosed herein and hereafter. In addition, when the feedstock is essentially free from insoluble solids with a particle size of at least 1.0 μm, at least 0.5 μm, or at least 0.2 μm, collecting of formed biomass is easier since insoluble solids with a particle size of at least 1.0 μm, at least 0.5 μm, or at least 0.2 μm may negatively clog the collecting apparatus or have a negative impact on filtering properties of the biomass. In addition, the collected formed biomass lacks insoluble solids that may be toxic or may bring undesirable properties to the biomass.

Alternatively, or additionally, methods for the continuous preparation of a fungal removing, biomass comprise ii) at least partially, insoluble solids from the feedstock, preferably wherein the insoluble solids have a size of at least 1.0 μm, more preferably at least 0.5 μm, even more preferably at least 0.2 μm. More preferably, removing insoluble solids having a size of at least 1.0 μm, preferably at least 0.5 μm, more preferably at least 0.2 μm, from the feedstock, preferably removing >50%, >70%, >80%, >90%, >95%, >97%, or >99% of the solids that are insoluble in the feedstock from the feedstock. Even more preferably, removing 100% of the solids that are insoluble in the feedstock from the feedstock.

Methods comprising step ii) enable the continuous preparation of fungal biomasses, wherein the crude protein content of step vi) is at least 60%, preferably 60-73%, 63-73%, 63-70%, or 65-70%, based on the total dry weight of the fungal biomass. Preferably, removing is selected from the group consisting of filtering, clarificating, decantating, settling, centrifugating, and screening. Preferably, after removing, at least partially, insoluble solids from the feedstock, the feedstock is essentially free from insoluble solids that have a size of at least 1.0 μm, more preferably at least 0.5 μm, more preferably at least 0.2 μm. As disclosed above, removing insoluble solids from the feedstock may form a biomass with a high crude protein content, may improve the collecting of formed biomass, and may eliminate potential toxic, harmful and/or unwanted insoluble solids ending up in the formed biomass.

Additionally, or alternatively, methods for the continuous preparation of a fungal biomass comprise iii) diluting or concentrating the feedstock, preferably diluting the feedstock. More preferably, diluting is adding water to the feedstock. It has surprisingly been found that by diluting or concentrating the feedstock comprising the one or more dissolved carbon sources to a carbon sources content of 1-10 wt %, preferably 2-4 wt %, more preferably 2-3 wt %, the formed fungal biomass has a crude protein content of at least 57% based on the total dry weight of the fungal biomass. In addition, it has surprisingly been found that by adjusting the concentration of carbon sources of the feedstock the productivity (g (protein) L⁻¹ h⁻¹) of the formed biomass may be increased, without wasting carbon sources.

Alternatively, or additionally, methods for the continuous preparation of a fungal biomass comprise after, before, or during vi):

adding a fresh feedstock comprising one ore more dissolved carbon sources to the combined one or more filamentous fungi and the feedstock.

It is to be understood that typically in methods for the continuous preparation of a fungal biomass the volume of fresh feedstock being added in vii) may be the same as the volume of the formed fungal biomass being collected in vi). E.g., when the dilution rate is 0.3 h⁻¹ in a method for the continuous preparation of a fungal biomass as disclosed herein and hereafter, fresh feedstock is being added in vii) having a volume that may correspond to 0.3 times the volume of the combined feedstock and the one or more filamentous fungi in iv) every hour, and formed fungal biomass is being collected from the feedstock in vi) having a volume that may be 0.3 times the volume of the combined feedstock and the one or more filamentous fungi in iv) every hour, therefore, the culture volume, i.e. the total volume of feedstock (including fresh feedstock) and one or more filamentous fungi, may be constant. Preferably, the fresh feedstock comprising one or more dissolved carbon sources is essentially free from insoluble solids. Alternatively, prior to vii) insoluble solids are removed, at least partially, from the fresh feedstock. It is to be understood that said fresh feedstock (or aqueous culture medium) may be feedstock (or aqueous culture medium), wherein the one or more filamentous fungi has not been cultivated in.

Alternatively, or additionally, the method for the continuous preparation of a fungal biomass is a method for the continuous preparation of a fungal biomass as disclosed herein and hereafter.

Alternatively, or additionally, the method for the continuous preparation of a fungal biomass further comprises sterilizing the feedstock prior to combining one or more filamentous fungi each independently selected from the fungal family Trichocomaceae with the feedstock.

Alternatively, or additionally, the crude protein content of step vi) is 60-73% based on the total dry weight of the fungal biomass.

Alternatively, or additionally, the dilution rate is selected from 0.30-0.45 h⁻¹. Alternatively, or additionally, the dilution rate is 0.30-0.41 h⁻¹. Alternatively, or additionally, the dilution rate is 0.35-0.41 h⁻¹. Alternatively, or additionally, the dilution rate is 0.30-0.35 h⁻¹. Alternatively, or additionally, the dilution rate is 0.30, 0.31, 0.35, 0.38, 0.40, 0.41, 0.45, or 0.50 h⁻¹. It has surprisingly been found that these dilution rates in continuous preparations of fungal biomasses according to methods disclosed herein and hereafter form fungal biomasses having higher crude protein content, i.e. a crude protein content of at least 57% based on the total dry weight of the fungal biomass. It has to be understood that the dilution rate may be changed during the method for continuous preparation of the fungal biomass. For example, first a dilution rate of 0.41 h⁻¹ may be used and subsequently a dilution rate of 0.31 h⁻¹ may be used, or first a dilution rate of 0.35 h⁻¹ may be used and subsequently a dilution rate of 0.41 h⁻¹ may be used.

Alternatively, or additionally, after optionally removing insoluble solids from the feedstock and/or optionally diluting or concentrating the feedstock, the one or more dissolved carbon sources content of the feedstock is 1-10 wt %, preferably 2-4 wt %, more preferably 2-3 wt %. It has surprisingly been found that using a feedstock having a one or more dissolved carbon sources content of 1-10 wt %, preferably 2-4 wt %, more preferably 2-3 wt %, the crude protein content of the formed fungal biomass is increased, wherein the crude protein content of the formed fungal biomass is at least 57, preferably 60-73%, based on the total dry weight of the fungal biomass. In addition, it has surprisingly been found that the effect of the one or more dissolved carbon sources content being 1-10 wt %, preferably 2-4 wt %, more preferably 2-3 wt %, is the carbon sources being effectively utilized by the one or more filamentous fungi in a method as disclosed herein and hereafter. Carbon sources contents >10 wt % may not be utilized by the one or more filamentous fungi, thereby wasting carbon sources and making the preparation method less effective/profitale, i.e. decreasing productivity of biomass. Carbon sources contents <1 wt % may be a limiting factor in the cultivation to form fungal biomass, thereby negatively affecting the productivity (g (protein) L⁻¹ h⁻¹) of the formed biomass.

Alternatively, or additionally, the one or more filamentous fungi is each independently selected from the fungal genera Paecilomyces, Gliocladium, Trichoderma, Byssochlamys, Spicaria, Aspergillus, Penicillium, Rasamsonia, Talaromyces, and Thermoascus.

Alternatively, or additionally, the one or more filamentous fungi is each independently selected from the fungal species Paecilomyces variotii, Paecilomyces puntonii, Gliocladium virens, Trichoderma viride, Byssochlamys nivea, Spicaria divaricata, Aspergillus niger, and Aspergillus oryzae.

Alternatively, or additionally, the one or more filamentous fungi is Paecilomyces variotii strain KCL-24. It has surprisingly been found that using Paecilomyces variotii strain in a KCL-24 method disclosed herein and hereafter fungal biomass is formed that has a high crude protein content, i.e. a crude protein content of at least 57% based on the total dry weight of the fungal biomass. In addition, Paecilomyces variotii strain KCL-24 forms fungal biomass with a suitable amino acid composition for foodstuff, a good protein digestibility in animals, and lack mycotoxins. Furthermore, it has surprisingly found that Paecilomyces variotii strain KCL-24 is suitable in a method for continuous preparation of fungal biomasses, wherein the dilution rate is 0.3-0.5 h⁻¹. The strain also enables a high productivity of fungal biomass and collecting the fungal biomass formed by Paecilomyces variotii strain KCL-24 is easier than biomass of non-filamentous fungi.

Alternatively, or additionally, the feedstock is selected from a thin stillage, vinasse, spent sulphite liquor, prehydrolysis liquor, food industry processing waste, and biorefinery by-product, or any mixture or combination thereof.

Alternatively, or additionally, at least one of the one or more dissolved carbon sources is each independently selected from carbohydrates, carbohydrate derivatives, sugars, oligosaccharides, polysaccharides, polyols, carboxylic acids, sugar acids, and alcohols, or any combinations thereof.

Alternatively, or additionally, the one or more dissolved carbon sources is each independently selected from carbohydrates, carbohydrate derivatives, sugars, oligosaccharides, polysaccharides, polyols, carboxylic acids, sugar acids, and alcohols, or any combinations thereof.

Alternatively, or additionally, the carbohydrates is each independently selected from glucose, mannose, xylose, arabinose, galactose, fructose, sucrose, maltose, isomaltulose, trehalose, lactose, maltotriose, maltodextrins, xylooligosaccharides (XOS), raffinose, stachyose, fructo-oligosaccharides; the polyols is each independently selected from glycerol, mannitol, sorbitol; the carboxylic acids is each independently selected from formic acid, acetic acid, lactic acid, propionic acid, aldonic acid, ulosonic acids, uronic acid, aldaric acid; the alcohols is each independently selected from methanol, ethanol, and ethylene glycol; or any combinations thereof. Preferably, the one or more dissolved carbon sources is each independently selected from sucrose, glucose, fructose, maltodextrins, xylose, mannose, glycerol, acetic acid, lactic acid, and formic acid, or combinations thereof.

Alternatively, or additionally, the method further comprises drying the fungal biomass after step vi). Alternatively, or additionally, the drying is performed in two or more steps each independently selected from drying using hot air, filtering, freeze drying, indirect or contact drying; and natural air drying, or any combination thereof. Preferably, the drying is filtering and drying hot using air. Preferably, the fungal biomass is dried until the fungal biomass has a water content of 0.5-10 wt %, preferably 3-8 wt %, more preferably 4-7 wt %, based on the total weight of the fungal biomass.

Alternatively, or additionally, the aqueous culture medium or the feedstock further comprises one or more organic and/or inorganic compounds each independently selected from the group consisting of nitrogen supplementation compounds, antifoaming agents, phosphorus supplementation compounds, trace elements, and inorganic salts. In embodiments, the one or more organic and/or inorganic compounds is each independently selected from the group consisting of NH₄OH, (NH₄)₂SO₄, CH₄N₂O, (NH₄)₂HPO₄, H₃PO₄, phosphates, KCl, MgSO₄, Fe₂(SO₄)₃, Fe(NH₄)₂(SO₄)₂, ZnSO₄, CuSO₄, MnSO₄, HCl, H₃BO₄, Na₂MoO₄, Vogel's trace elements, Struktol J673A, citric acid, and any salts, hydrates and combinations thereof.

Alternatively, or additionally, the method for the continuous preparation of a fungal biomass further comprises after, before, or during iv), v), and/or vi):
adding one or more organic and/or inorganic compounds each independently selected from the group consisting of nitrogen supplementation compounds, antifoaming agents, phosphorus supplementation compounds, trace elements, and inorganic salts to the aqueous culture medium or the feedstock.

In embodiments, the one or more organic and/or inorganic compounds are each independently selected from the group consisting of NH₄OH, (NH₄)₂SO₄, CH₄N₂O, (NH₄)₂HPO₄, H₃PO₄, phosphates, KCl, MgSO₄, Fe₂(SO₄)₃, Fe(NH₄)₂(SO₄)₂, ZnSO₄, CuSO₄, MnSO₄, HCl, H₃BO₄, Na₂MoO₄, Vogel's trace elements, Struktol J673A, citric acid, and any salts, hydrates and combinations thereof.

Alternatively, or additionally, the feedstock comprising one or more dissolved carbon sources is selected from thin stillage, molasses, vinasses, and other feedstocks comprising one or more dissolved carbon sources, wherein the one or more dissolved carbon sources comprise at least one carbon source each independently selected from the group consisting of glycerol, acetic acid, formic acid, sucrose, glucose, fructose, maltodextrins, xylose, mannose, and lactic acid, or any combinations or mixtures thereof; wherein the feedstock further comprises (NH₄)₂SO₄ and an antifoaming agent, preferably, Struktol J673A.

Alternatively, or additionally, the method further comprises adjusting the pH of the combined one or more filamentous fungi each independently selected from the fungal family Trichocomaceae and feedstock. Preferably, the pH is adjusted to 3.0-6.0, more preferably to 4.5-5.0.

Alternatively, or additionally, the cultivating the combined the one or more filamentous fungi and the feedtsock is perfomed at 30-45° C., preferably at 35-41° C., more preferably at 37-39° C. Alternatively, or additionally, the aerobic conditions comprise an aeration rate of 0.1-1.0 volume per volume per minute (VVM), preferably 0.1-0.6 VVM, more preferably 0.1-0.3 VVM.

Alternatively, or additionally, the combined the one or more filamentous fungi and the feedstock is mixed during the cultivation.

Alternatively, or additionally, the method further comprises fractionating the fungal biomass after step vi) to form one ore more fractions. Alternatively, or additionally, the one or more fractions is at least a fungal protein fraction and/or a β-glucan fraction.

Alternatively, or additionally, the cell density of the one or more filamentous fungi is 5-20 g/L during the cultivating the combined the one or more filamentous fungi and the feedstock. The cell density of 5-20 g/L enables easier harvesting/collecting of the formed fungal biomass from the feedstock.

Alternatively, or additionally, the feedstock is comprising two or more feedstocks each comprising one or more dissolved carbon sources, wherein the two or more feedstocks are each independently selected from the group consisting of thin stillage, vinasse, spent sulphite liquor, prehydrolysis liquor, food industry processing waste, and biorefinery by-product, or any mixture or combination thereof, wherein the carbon sources are each independently selected from the group consisting of carbohydrates, carbohydrate derivatives, sugars, oligosaccharides, polysaccharides, polyols, carboxylic acids, sugar acids, and alcohols, or any combinations thereof.

Alternatively, or additionally, the feedstock is selected from thin stillage, vinasse, spent sulphite liquor, prehydrolysis liquor, food industry processing waste, and biorefinery by-product, or any mixture or combination thereof; the feedstock comprises one or more dissolved carbon sources each independently selected from glycerol, acetic acid, formic acid, sucrose, glucose, fructose, maltodextrins, xylose, mannose, and lactic acid, or combinations thereof; wherein the feedstock comprising one or more dissolved carbon sources is essentially free from insoluble solids; wherein the one or more dissolved carbon sources content of the feedstock is 2-6 wt %; the cell density of the one or more filamentous fungi is 5-20 g/L, and the dilution rate is 0.30-0.35 h⁻¹.

In embodiments are provided methods for the continuous preparation of a fungal biomass, comprising:
i) providing a feedstock comprising one or more dissolved carbon sources;
iv) combining one e or more filamentous fungi each independently selected from the fungal family Trichocomaceae with the feedstock;
v) cultivating the combined one or more filamentous fungi and the feedstock under aerobic conditions to form the fungal biomass;
vi) collecting the formed fungal biomass from the feedstock when the formed fungal biomass has a crude protein content of at least 57% based on the total dry weight of the fungal biomass,
   wherein the dilution rate is 0.3-0.5 h⁻¹, wherein the feedstock is selected from stillage, thin stillage, vinasse, molasses, spent sulphite liquor, prehydrolysis liquor, food industry processing waste, and biorefinery by-products, or any mixture or combination thereof, and wherein the
   one or more dissolved carbon sources comprise at least one or more organic compounds each independently selected from glucose, mannose, xylose, arabinose, galactose, fructose, sucrose, maltose, isomaltulose, trehalose, lactose, maltotriose, maltodextrins, xylooligosaccharides (XOS), raffinose, stachyose, fructo-oligosaccharides, glycerol, mannitol, sorbitol, formic acid, acetic acid, lactic acid, propionic acid, methanol, ethanol, ethylene glycol, N-acetylglucosamineglucose, saccharose, and glycerol, wherein the one or more dissolved carbon sources content of the feedstock is 2-4 wt %, preferably 2-3 wt %, wherein the feedstock comprising one or more dissolved carbon sources is essentially free from insoluble solids, and wherein collecting the formed fungal biomass from the feedstock when the formed fungal biomass has a crude protein content of 60-73%, 63-73%, 63-70%, 65-73%, or 65-70%, based on the total dry weight of the fungal biomass.

Additionally, or alternatively, vi), in collecting the formed fungal biomass from the feedstock when the formed fungal biomass has a crude protein content of at least 61%, or 61-70%, 61-73%, 61-77%, 61-81%, 61-85%, 60-89%, 60-93%, 60-95%, or 60-99%, based on the total dry weight of the fungal biomass.

Additionally, or alternatively, in i), the one or more dissolved sources content of the feedstock is 1-10 wt %; and in v), the temperature during the cultivating is 30-45° C., the cell density of the one or more filamentous fungi during the cultivating is 5-20 g/L, the aerobic conditions comprise an aeration rate of 0.1-1.0 VVM, and the pH of the combined one or more filamentous fungi and feedstock during the cultivating is 3.0-6.0, preferably further wherein the cultivating is performed at least until steady state of the one or more filamentous fungi has been reached.

In one aspect is disclosed edible compositions comprising fungal biomass as disclosed herein hereafter. In embodiments the edible composition comprising fungal biomass as disclosed herein and hereafter is fish feed or aquafeed, preferably aquafeed. Edible compositions as disclosed herein and hereafter comprising fungal biomass as disclosed herein and hereafter are beneficial, since all, or at least partially, the SPC aquafeed fish that and feed conventially comprises, may by replaced by the biomass as disclosed herein and hereafter.

Additionally, provided are edible compositions comprising fungal biomass as disclosed herein and hereafter combined with one or more food ingredient, preferably the edible composition is a fish feed or aquafeed, more preferably aquafeed.

Additionally, or alternatively, the edible composition is aquafeed and the composition comprises protein originating only from fungal biomass as disclosed herein and hereafter.

Additionally, or alternatively, the one or more food ingredient is each independently selected from the group consisting of fishmeal, fish oil, fish feed, soy protein concentrate, soy, soybeans, wheat protein, pea protein, soy protein isolate, wheat protein isolate, pea protein isolate, corn, grain sorghum, oats, rye, barley, food additives, flours, and dairy products, or any combinations thereof.

In embodiments, the food additive is selected from the group consisting of acidulants, acidity regulators, anticaking agents, antifoaming and foaming agents, antioxidants, bulking agents, food coloring, fortifying agents, color retention agents, emulsifiers, flavors, flavor enhancers, flour treatment agents, glazing agents, humectants, tracer gas, preservatives, stabilizers, sweeteners, and thickeners, or any combination thereof.

In embodiments, the flour is selected from the group consisting of wheat flour, rye flour, fishmeal, farina, and meal, or any combination thereof.

In embodiments, the dairy product is selected from the group consisting milk, yoghurt, curdled milk (soured milk), and cheese, or any combinations thereof.

Additionally, or alternatively, the edible composition comprises 10-30 wt % of fungal biomass as disclosed herein and hereafter, preferably 15-27 wt %, more preferably 26-27 wt %, and 70-80 wt % one or more food ingredient, provided that when the one or more food ingredient comprises soy protein and/or soy protein concentrate (SPC), the total content of soy protein and/or SPC is 0-20 wt %, preferably 0-4 wt %, more preferably 0 wt %.

Additionally, or alternatively, the edible composition comprises 15-27 wt % of the fungal biomass, 9-11 wt % of fishmeal, 4-7 wt % of water, and soy protein and/or SPC, wherein the content of soy protein and/or SPC is 0-12 wt %, preferably 0-4 wt %, more preferably 0 wt %.

Additionally, or alternatively, the edible composition comprises 20-30 wt %, preferably 26-27 wt %, of the fungal biomass, 0-11 wt % fishmeal, faba beans 0-5 wt %, wheat gluten 0-12 wt %, sunflower meal 0-1.5 wt %, guar meal 0-3 wt %, fish oil from whole fish 0.5-9.5 wt %, fish oil from trimmings 0-1.5 wt %, micro algal oil 0-0.15 wt %, fish oil from farmed fish 0-0.8 wt %, rapeseed oil 15-25 wt %, camelina oil 0-1.5 wt %, wheat 6-10 wt %, carbohydrates 0-4.6 wt %, and SPC 0-7 wt %, preferably 0-4 wt %, more preferably 0 wt %.

In one aspect is disclosed uses of a fungal biomass as disclosed herein or an edible composition as disclosed herein as or in foodstuff.

In embodiments is provided use of a fungal biomass as disclosed herein or an edible composition as disclosed herein wherein the foodstuff is selected from the group consisting of food for consumption of humans, compound feed, fodder, and animal feed.

In embodiments is provided use of a fungal biomass as disclosed herein or an edible composition as disclosed herein in foodstuff, wherein the foodstuff is fish feed or aquafeed.

In another aspect is disclosed uses of a fungal biomass as disclosed herein or an edible composition as disclosed herein as a food ingredient in foodstuff. In embodiments is provided use of a fungal biomass as disclosed herein or an edible composition as disclosed herein as a protein source in fish feed or aquafeed.

In another aspect is disclosed uses of a fungal biomass as disclosed herein or an edible composition as disclosed herein as a replacement for soy protein or SPC in foodstuff. In embodiments the foodstuff is aquafeed or fish feed.

In another aspect is disclosed use of a fungal biomass disclosed herein to purify a feedstock comprising one or more dissolved carbon sources, wherein the one or more dissolved carbon sources are, at least partially, removed from the feedstock.

### EXAMPLES

Reference will now be made in detail to various embodiments.

The description below discloses some embodiments in such a detail that a person skilled in the art is able to utilize the embodiments based on the disclosure. Not all steps or features of the embodiments are discussed in detail, as many of the steps or features will be obvious for the person skilled in the art based on this specification.

### Example 1: Inoculum Preparation

Trichocomaceae fungus Paecilomyces variotii strain KCL-24 was obtained from the VTT Culture Collection. The cultures were maintained on potato dextrose agar (PDA) plates. For preparing mycelium suspension, the mycelium of Trichocomaceae fungus Paecilomyces variotii strain KCL-24 was aseptically released using a disposable cell spreader and suspended into 20 mL of sterile water. To prepare fungal biomass inoculum, 5 mL of the mycelium suspension was inoculated into 50 mL of standard medium (table 1). The culture was incubated in a 250-mL shake flask for 24 h at 37° C. and 250 rpm and used as the inoculum in methods for the continuous preparation of fungal biomasses.

TABLE 1
Content of standard medium

| Substance | Amount |
|---|---|
| (NH₄)₂SO₄ | 3.5 g/L |
| NH₄OH (25%) | 4 mL/L |
| H₃PO₄ (85%) | 0.6 mL/L |
| KCl | 0.15 g/L |
| MgSO₄*7H₂O | 0.15 g/L |
| Vogel's trace | 0.1 mL/L |
| elements (10 000×) | |
| Glucose | 25 g/L |
| Molasses¹ | 5 g/L |

¹Typically, molasses comprises ca.50 wt % sugars, typically saccharose.

### Example 2: General Method for Continuous Preparations of a Fungal Biomasses

To prepare a continuous bioprocess (continuous preparation) of fungal biomass in a bioreactor, the inoculum (50 mL) prepared in example 1 was aseptically inoculated into 2.95 L of production medium (feedstock) in a stirred tank bioreactor and incubated for 24 h at 37° C., 1200 rpm, aeration rate 0.3 VVM. The production medium contained the feedstock in question as is or as diluted, nitrogen supplementation with (NH₄)₂SO₄, an antifoaming agent such as Struktol J673A, and, in some cases, phosphorus supplementation with H₃PO₄. After the incubation, the continuous bioprocess was initiated by starting the feeding of production medium (i.e. fresh feedstock) to the bioreactor and collecting suspension comprising formed fungal biomass form the bioreactor at the same rate called the dilution rate. The aeration rate was kept at 0.3 VVM. The collected suspension comprising fungal biomass was dewatered (dried) in two steps, first by filtering and subsequently by drying using hot air (50° C.).

### Example 3: Methods for Continuous Preparations of Fungal Biomasses Using Different Dilution Rates

Method of example 2 was used and the standard medium (table 1) was used as the production medium, i.e., as feedstock and fresh feedstock. Dilution rates 0.07-0.41 h⁻¹ were used in this series of parallel continuous preparations of fungal biomasses. Increasing the dilution rate results in increased crude protein content of the fungal biomass (table 2). Using the dilution rate 0.41 h⁻¹ resulted in a crude protein content (total dry weight basis) of 70% of the fungal biomass (table 2).

Table 2. Examples of crude protein contents (%) of fungal biomasses obtained with different dilution rates (h⁻¹) in continuous preparations of fungal biomasses. Increasing the dilution rate results in higher crude protein content of the fungal biomass.

| Dilution rate (h⁻¹) | Crude protein content (%) |
|---|---|
| 0.07 | 50.9 ± 0.1 |
| 0.14 | 54.9 ± 1.0 |
| 0.17 | 56.0 ± 0.2 |
| 0.24 | 59.5 ± 0.2 |
| 0.27 | 62.6 ± 0.7 |
| 0.31 | 64.6 ± 1.6 |
| 0.38 | 66.1 |
| 0.41 | 69.5 ± 0.1 |

### Example 4: Thin Stillage as Feedstocks and Different Dilution Rates in Continuous Preparations of Fungal Biomasses

The method of example 2 was used and as the production medium (i.e., as feedstock and fresh feedstock) unconcentrated thin stillage from corn bioethanol process was used; the unconcentrated thin stillage comprising 3.5 wt % carbon sources of which about 50% is glycerol and having in total 5.5 wt % dissolved solids, and the production medium (as feedstock and fresh feedstock) was diluted with water with a 1:1 ratio, and was supplemented with 5 g/L of (NH₄)₂SO₄ and 0.5 mL/L of Struktol J673A. In this series of parallel continuous preparations of fungal biomasses dilution rates 0.3 h⁻¹, 0.35 h⁻¹, and 0.4 h⁻¹ were used and the crude protein contents (total dry weight basis) were determined. Increasing the dilution rate results in higher crude protein content of the fungal biomass (table 3).

Table 3. Examples of obtained crude protein contents (%) of fungal biomasses obtained using thin stillage as the feedstock and fresh feedstock and different dilution (h⁻¹) rates in continuous preparations of fungal biomasses. Increasing the dilution rate results in higher crude protein content of the fungal biomass.

| Dilution rate (h⁻¹) | Crude protein content % |
|---|---|
| 0.30 | 62.5 ± 1.2 |
| 0.35 | 63.2 ± 0.3 |
| 0.40 | 65.2 ± 0.1 |

### Example 5: Non-Clarified and Clarified Vinasse as Feedstocks and Different Dilution Rates in Continuous Preparations of Fungal Biomasses

The method of example 2 was used and in this series of parallel continuous preparations of fungal biomasses as the production medium (feedstock and fresh feedstock) was used unconcentrated non-clarified or clarified vinasse from sugar beet ethanol process; the unconcentrated non-clarified and clarified vinasses comprising 2.8 wt % carbon sources of which about 40% is glycerol and having in total 4.8 wt % dissolved solids, and the production medium (as feedstock and fresh feedstock) was diluted with water with a 1:1 ratio and was supplemented with 5 g/L of (NH₄)₂SO₄, 0.6 mL/L of H₃PO₄, and 0.5 ML/L of Struktol J673A. The clarified vinasse was obtained by decanting unconcentrated non-clarified vinasse, and solids that are insoluble in the non-clarified vinasse and with a size of at least 1.0 μm had been removed, at least partially, from the non-clarified vinasses. The dilution rate 0.35 h⁻¹ was used in all of the parallel continuous preparations and the crude protein contents (total dry weight basis) were determined. Using clarified vinasse as the feedstocks results in higher crude protein content of the fungal biomass (table 4).

Table 4. Examples of crude protein contents (%) of fungal biomass obtained using unconcentrated non-clarified or clarified vinasse from sugar beet ethanol process as the feedstock and fresh feedstock in continuous preparations of fungal biomasses.

| Feedstocks used in | Dilution | Crude protein |
|---|---|---|
| the methods | rate (h⁻¹) | content (%) |
| Non-clarified vinasse | 0.35 | 63.3 ± 0.8 |
| Clarified vinasse | 0.35 | 67.1 ± 0.6 |

### Example 6: Non-Diluted and Diluted Thin Stillage as Feedstocks in the Continuous Preparations of Fungal Biomasses

The method of example 4 was used except that the unconcentrated thin stillage was used without dilution or the unconcentrated thin stillage was diluted with water to a concentration of 67% (i.e., diluted with a 2:1 (unconcentrated thin stillage:water) ratio) or 50% (i.e., diluted with a 1:1 (unconcentrated thin stillage:water) ratio), and the feedstocks were supplemented with 5 g/L of (NH₄)₂SO₄ and 0.5 mL/L of Struktol J673A. In this series of parallel continuous preparations of fungal biomasses, the dilution rate 0.3 h⁻¹ was used and the yields in grams of cells of biomasses produced per liter of 100% thin stillage (total dry weight basis) were determined. Diluting the thin stillage results in lower content of carbon sources, which results in higher yields of the biomasses and lower production costs of the biomasses useful as a protein source in foodstuff (table 5).

Table 5. Examples of cell yields (g cells/L 100% thin stillage) of fungal biomass obtained using different thin stillage concentrations (%) as the feedstock and fresh feedstock in continuous preparations of fungal biomasses.

| Thin stillage | |
|---|---|
| concentration | Yield |
| in the feedstocks | (g cells/L 100% thin stillage) |
| 100% | 10.2 ± 0.5 |
| 67% | 14.0 ± 0.4 |
| 50% | 18.3 ± 0.6 |

### Example 7: Continuous Preparations of Fungal Biomasses Using Aspergillus oryzae

To prepare a continuous bioprocess (continuous preparation) of fungal biomass in a bioreactor using Aspergillus oryzae (strain number D-88355T), an inoculum (50 mL) was prepared according to example 1 except that Trichocomaceae fungus s Aspergillus oryzae strain D-88355T, obtained from the VIT Culture Collection, was used. To prepare the fungal biomass inoculum, 5 mL of the mycelium suspension was inoculated into 50 mL of standard medium (table 6). The culture was incubated in a 250-mL shake flask for 24 h at 30° C. and 250 rpm and used as the inoculum in methods for the continuous preparation of fungal biomasses using Aspergillus oryzae.

TABLE 6
Content of standard medium.¹

| Substance | Amount |
|---|---|
| (NH₄)₂SO₄ | 2.0 g/L |
| NH₄)₂HPO₄ | 2.0 g/L |
| MgSO₄*7H₂O | 0.15 g/L |
| Vogel's trace | 0.1 mL/L |
| elements (10 000×) | |
| Structol J673A | 0.20 g/L |
| Molasses² | 38.6 g/L |

¹NH₄OH (25% (v/v) in H₂O) and H₃PO₄ (25 wt % in H₂O) were used to adjust the pH of the standard medium to 5.2.
²Typically, molasses comprises ca.50 wt % sugars, typically saccharose.

The method of example 2 was followed except that the standard medium (table 6) was used as the production medium, i.e., as feedstock and fresh feedstock. In addition, dilution rates 0.10-0.30 h⁻¹, aeration rate of 0.3 VVM, a temperature of 30° C., NH₄OH (25% (v/v) in H₂O) and H₃PO₄ (25 wt % in H₂O) were used to maintain the pH at 5.2 during the fermentation processes, and stirring at 800-1000 rpm were used in this series of parallel continuous preparations of fungal biomasses. The collected suspension comprising fungal biomass was dewatered (dried) in two steps, first by filtering and subsequently by drying using hot air (50° C.). Increasing the dilution rate results in increased crude protein content of the fungal biomass (table 7). Using the dilution rate 0.30 h⁻¹ resulted in a crude protein content (total dry weight basis) of ca. 61% of the fungal biomass (table 7).

Table 7. Examples of crude protein contents (%) of fungal biomasses obtained with different dilution rates (h⁻¹) in continuous preparations of fungal biomasses using Aspergillus oryzae.

| Dilution rate (h⁻¹) | Crude protein content (%) |
|---|---|
| 0.10 | 39.0 |
| 0.15 | 40.4 |
| 0.30 | 60.9 |

It is obvious to a person skilled in the art that with the advancement of technology, the inventive concept can be implemented in various ways. The invention and its embodiments are thus not limited to the examples described above; instead they may vary within the scope of the claims.

The embodiments described hereinbefore may be used in any combination with each other. Several of the embodiments may be combined together to form a further embodiment. A product, a system, a method, or a use, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages.

1. A fungal biomass comprising one or more filamentous fungi each independently selected from the fungal family Trichocomaceae, wherein the fungal biomass has a crude protein content of at least 61% based on the total dry weight of the fungal biomass.

2. The fungal biomass as claimed in claim 1, wherein the fungal biomass has a crude protein content of 61-70%, 63-70% or 66-70% based on the total dry weight of the fungal biomass.

3. The fungal biomass as claimed in claim 1, wherein the one or more filamentous fungi is each independently selected from the fungal genera Paecilomyces, Aspergillus, Penicillium, Rasamsonia, Talaromyces, and Thermoascus.

4. The fungal biomass as claimed in claim 1, wherein the one or more filamentous fungi is each independently selected from the fungal species Paecilomyces variotii, Paecilomyces puntonii, Aspergillus niger, and Aspergillus oryzae.

5. The fungal biomass as claimed in claim 1, wherein the one or more filamentous fungi is Paecilomyces variotii strain KCL-24.

6. The fungal biomass as claimed in claim 1, wherein the fungal biomass comprises 0.5-10 wt % water, a total fiber content of 10-35 wt %, a β-glucan content of 10-25 wt %, 1-10 wt % ash, and 1-10 wt % fat based on the total weight of the fungal biomass.

7. The fungal biomass as claimed in claim 1, wherein the fungal biomass has a water content of 3-8 wt %, preferably 4-7 wt %, based on the total weight of the fungal biomass.

8. The fungal biomass as claimed in claim 1, wherein the fungal biomass is essentially free from solids that are insoluble in a feedstock comprising one or more dissolved carbon sources.

9. The fungal biomass as claimed in claim 1, wherein the fungal biomass is edible.

10. A method for the continuous preparation of a fungal biomass, comprising:
i) providing a feedstock comprising one or more dissolved carbon sources;
ii) optionally removing, at least partially, insoluble solids from the feedstock;
iii) optionally diluting or concentrating the feedstock;
iv) combining one or more filamentous fungi each independently selected from the fungal family Trichocomaceae with the feedstock;
v) cultivating the combined one or more filamentous fungi and the feedstock under aerobic conditions to form the fungal biomass;
vi) collecting the formed fungal biomass from the feedstock when the formed fungal biomass has a crude protein content of at least 61% based on the total dry weight of the fungal biomass,
wherein the dilution rate is 0.3-0.5 h⁻¹.

11. The method as claimed in claim 10, wherein the crude protein content of the fungal biomass in step vi) is 61-70% based on the total dry weight of the fungal biomass.

12. The method as claimed in claim 10, wherein after optionally removing insoluble solids from the feedstock and/or optionally diluting or concentrating the feedstock, the one or more dissolved carbon sources content of the feedstock is 1-10 wt %, preferably 2-4 wt %, more preferably 2-3 wt %.

13. The method as claimed in claim 10, wherein the one or more filamentous fungi is each independently selected from the fungal genera Paecilomyces, Aspergillus, Penicillium, Rasamsonia, Talaromyces, and Thermoascus.

14. The method as claimed in claim 10, wherein the one or more filamentous fungi is each independently selected from the fungal species Paecilomyces variotii, Paecilomyces puntonii, Aspergillus niger, and Aspergillus oryzae.

15. The method as claimed in claim 10, wherein the one or more filamentous fungi is Paecilomyces variotii strain KCL-24.

16. The method as claimed in claim 10, wherein the feedstock is selected from a thin stillage, vinasse, spent sulphite liquor, prehydrolysis liquor, food industry processing waste, and biorefinery by-product, or any mixture or combination thereof.

17. The method as claimed in claim 10, wherein the one or more dissolved carbon sources is each independently selected from carbohydrates, carbohydrate derivatives, polyols, carboxylic acids, amino acids, and alcohols, or any combinations thereof.

18. The method as claimed in claim 10, further comprising drying the fungal biomass after step vi).

19. The method as claimed in claim 10, wherein the cell density of the one or more filamentous fungi is 5-20 g/L during the cultivating the combined the one or more filamentous fungi and the feedstock.

20. An edible composition comprising fungal biomass as defined in claim 1.

21. Use of a fungal biomass as defined in claim 1 or an edible composition as defined in claim 20 as or in foodstuff.
